(19) United States
(12) Patent Application Publication
(10) Pub. No.: US 2012/0108629 A1
(43) Pub. Date: May 3, 2012

(54) New use of neferine

(75) Inventors: Tingliang Jiang, Beijing (CN); Feng Sui, Beijing (CN); Hairu Huo, Beijing (CN); Lanfang Li, Beijing (CN); Shuying Guo, Beijing (CN)

(73) Assignee: Institute of Chinese Materia Medica, China Academy, Beijing (CN)

(21) Appl. No.: 13/264,375

(22) PCT Filed: Apr. 13, 2010
(86) PCT No.: PCT/CN2010/071721

§ 371 Date: Nov. 11, 2011

(30) Foreign Application Priority Data
Apr. 14, 2009 (CN) 200910082318.8

(51) Int. Cl. A61K 31/4725; A61P 25/00; A61P 25/06; A61P 25/18; A61P 11/04; A61P 11/06; A61P 19/02; C07D 217/04; A61P 11/14

(52) U.S. Cl. 514/308; 546/140

(57) ABSTRACT

The present invention reveals a new use of neferine. Neferine regulates M8 and V1 subtype of transient receptor potential ion channel (TRPM8 and TRPV1), respectively, in mammalians, including humans, and can be used for preparing medicaments for treating disease related to said ion channel (such as hyperalgesia, Parkinson's disease, painful bladder syndrome, chronic obstructive pulmonary disease, and tumor of skin, prostate, mammary gland, lung, colon, etc). The potency of neferine in the present invention is higher than that of hexahydrothymol.

### FIELD OF THE INVENTION

The present invention relates to a novel use of neferine which is a compound from Chinese herb. In particularly, the present invention relates to the use of neferine in the preparation of compounds for the regulation of M8 and V1 subtypes of Transient Receptor Potential ion channel (TRPM8 and TRPV1 hereinafter) and medicaments involved in the pathological processes and diseases associated with TRPM8 and TRPV1.

### BACKGROUND OF THE INVENTION

TRP is a kind of transmembrane structure on the cell membrane or intracellular organelles film recently discovered on mammals and human cell. It can feel numerous of extra or intra cell messages. Activation or block of TRP channels could cause opening or closure of these channels and thus induce entry into cytoplasm of extra-cellular calcium or excretion out of free calcium from intracellular stores. These signaling pathways have been recognized to be involved in many pathological processes by modifying the specific cell functions and thus TRP channels are regarded as potentially important novel targets for developing new drugs.

TRPM8 was first recognized as a kind of ion channels for the stimulated reaction of androgen in prostate. In 2002, McKemy et al. cloned this receptor channel and named it cold and menthol sensitive receptor 1 (CMR1), which belongs to the TRPM subfamily. It is found recently that TRPM8 can be activated, inhibited or regulated by one of physical, chemical or intracellular pH value factor. TRPM8 mainly distributed in tissues and organs such as small-size neurons of trigeminal and dorsal root ganglion (DRG), prostate epithelium, taste nipple, testis, seminiferous tubules, scrotum skin, bladder cell, bladder and urinary tract epithelium, lung, thymus, mammary gland and small intestine epithelium; as well as malignant tumor tissues such as melanoma, prostatic cancer, breast cancer, lung cancer and colorectal cancer. TRPM8 is involved in lots of pathogenesis of diseases such as hyperalgesia to cold, Parkinson's disease, painful bladder syndrome, chronic obstructive lung disease, and occurrence and development of skin, prostate, breast, lung, and colon cancers and the like. For example, hexahydrothymol can up-regulate TRPM8 and inhibit the proliferation of melanoma. The TRPM8 inhibitor N-(3-aminopropyl)-2-{[(3-methylphenyl) methyl]oxy}-N-(2-thienylmethyl) benzamide hydrochloride salt (AMTB) can relieve painful bladder syndrome.

TRPV1 is one type of channel protein in TRPV subfamily cloned by Juilin S, et al. in 1997. It has been named as capsaicin receptor or vanilloid receptor subtype 1(VR1), and has now become the most studied type in TRP superfamily. TRPV1 is extensively distributed in a multitude of organs and tissues, such as brain, cardiovascular system, lung, digestive, urogenital system and on the like. It is well recognized that over-expression or activation of TRPV1 can result in a wide array of pathological states such as hypersensitivity to heat, allodynia, migraine, toothache, vulvodynia, painful gastrointestinal diseases, esophagitis, gastroesophageal reflux disease, functional intestinal diseases, ulcerative colonitis, pancreatitis, cystitis and bladder hyper-reactivity, osteoarthritis and cough, chronic obstructive lung disease and asthma, allergic rhinitis, non-insulin-dependent diabetes, myasthenia syndrome, schizophrenia, and breast cancer. It is also involved in the regulation and control of tension and permeability of the blood vessel. Thus, there are lots of beneficial effects on the body, such as reducing reperfusion injury after ischemic insult, protection on brain and heart, as well as promotion of hearing threshold upon activation of TRPV1. It was reported that the potential drugs in investigation, as antagonists or agonists of TRPV1, such as capsaicin transdermal patch for HIV nervous pain, capcaicin injection for pain, and GRC621 oral preparation for bone and joint pain, are all now in clinical trial phase I-III.

Neferine, with the molecular weight of 624.78, is one of the main constituents of Chinese herb Plumula nelumbinis which has the effect of lowering blood pressure. Neferine can be extracted from Plumula nelumbinis directly or commercial available.

No reports about the effect of neferine on TRPM8 and/or TRPV1 were found through searching the public literature databases such as Pubmed, CNKI and so on.

### SUMMARY OF THE INVENTION

The present invention relates to the use of neferine in the preparation of compounds for the up-regulation of TRPM8 in mammalians including humans.

As neferine can up-regulate TRPM8, it can be used potentially in the preparation of medicaments for the prevention and/or treatment of diseases involved in TRPM8 such as chronic obstructive lung disease, Parkinson's disease, painful bladder syndrome, cold hyperalgesia, malignant tumors such as melanoma, prostate, breast and pancreas cancer.

This invention also relates to the use of neferine in the preparation of compounds for the down-regulation of TRPV1 in mammalians.

As a result of the fact that neferine can down-regulate TRPV1, it could be used in the preparation of medicaments for the prevention and treatment of diseases involved in TRPV1 such as the thermal hyperalgesia, allodynia, migraine, tooth pain, vulvodynia, painful gastrointestinal disease, esophagitis, gastro-esophageal reflux, functional bowel disorders, ulcerative colitis, pancreatitis, cystitis and hyper-reactivity of bladder, osteoarthritis, cough, chronic obstructive lung disease and asthma, allergic rhinitis, non-insulin-dependent diabetes, myasthenia syndrome, schizophrenia and breast cancer, pancreatic cancer and the like.

The compounds of the present invention may be further used in combination with various of pharmaceutically acceptable carriers commonly known in the art. The carriers include, for example, diluents, adhesives, absorbents, disintegrants, dispersant, wetting agent, auxiliary solvent, buffers, surfactants and the like.

The neferine of the present invention may be prepared into various of dosage forms respectively such as powder, liquid, and gas. The dosage forms may be suitable for intestinal tract or parenteral administration, such as oral, intramuscular, subcutaneous, nasal cavity, oral mucosa, skin, peritoneal and rectal administration.

The neferine of the present invention or the compositions thereof can be administrated singly by intestinal tract or parenteral, such as oral, intramuscular, subcutaneous, nasal cavity, oral mucosa, skin, peritoneal, and rectal injection.

On the condition that neferine can up-regulate TRPM8 and down-regulate TRPV1, the neferine may have an similar effect to TRPM8 agonists or TRPV1 antagonists.

The present invention thus relates to the use of neferine in the preparation of medicaments for the activation of TRPM8 and inhibition of TRPV1 in mammalians including humans.

TRPM8 is a kind of ion channels newly identified during the last decade. The roles of it in health and disease are gaining more and more attentions. In view of the fact that there were still neither available drugs on the market, nor reports about the development of any targeting-TRPM8 drugs, the present invention thus may be of great inventive step and potentially provide a new option for patients with cold hyperalgesia, Parkinson's disease, painful bladder syndrome, chronic obstructive lung disease, as well as skin, prostate, breast, lung and colon cancer and the like. Hexahydrothymol is a common known agonist for experimental use. The neferine of the present invention has a stronger effect than hexahydrothymol.

### DETAILED DESCRIPTION OF THE INVENTION

Obtaining and Treating compounds: Neferine with purity ≧90% was obtained from market and prepared into desired differing concentrations (μmol/L) according to requirements.

Preparation of rat DRG cells: Dorsal root ganglion (DRG) cell is the main afferent neurons in the mammalian and human peripheral nervous system. It expresses a wide array of TRP channel proteins and plays a key role in nerve protection, repair after nerve damage, transmission of noxious stimuli, pathological and physiological processes. To investigate the effect of neferine on TRPM8, newborn Sprague-Dawley rats (5 days after born) were selected. DRG were aseptically removed and meticulously cleaned the rootlets and connective tissue. The tissue was digested with collagenase and trypsin in succession, and then centrifuged and the digestive liquid was discarded. Cells were prepared into single cell suspension in common culture medium, and then calculated and adjusted to 10⁵ nerve cells per ml. It was incubated in culture plates pre-coated with laminin at 37° C. in 5% CO₂ for 48 hours. Camptothecine was used to inhibit proliferation of other cells. Culture was continued to obtain DRG cells with a high purity (>98%).

Detection of TRPM8 and TRPV1 mRNA expression: The total RNA of the DRG neuronal cells was extracted in a routine way, after exposure to the tested agents of various concentrations under the normal, low and high temperature conditions respectively. The concentration of RNA was measured and its cDNA was prepared. The desired amount of resulting cDNA and TRPM8 or TRPV1 gene primers were added on the IQ5-Type real-time IQ5-Type real-time PCR meter with GAPDH as the endogenous control to perform extension. Melting curves were generated. Relative gene expression was calculated using the comparative threshold cycle (2-^{ΔΔCT}) method with the calculated formula as following:

ΔΔCt=(Ct_{,Target}-C_{t,GAPDH})ₓ-(Ct_{,Target}-C_{t,GAPDH})_{Control}

X represents any one of tested, Control represents double target gene expression after GAPDH correction.

Detection of intracellular calcium concentrations of DRG cells: DRG cells cultured under normal, low and high temperature conditions was obtained. The medium was discarded and the cells were washed after certain time. 0.1%F-127, 5 μM of Fluo-4-AM, M fluorescent dye were added and loaded at 25° C. out of light for 30 min. After dye loading, the redundant dye was washed out and buffer and test compounds were added respectively. The cells were put in a closed chamber on a microscope stage. Fluorescence dyed cells were detected with appropriate conditions. The change of fluorescence intensity in each cell was determined and the data were analyzed by software. The change of the intracellular calcium concentration was indicated by fluorescence intensity. The change degree of the intracellular calcium concentration was represented by the percent ratio of the change value of fluorescence intensity before and after administration and the value of fluorescence intensity before administration (Δ[Ca2+]i), i.e. Δ[Ca2+]i=(fluorescence intensity value after administration F-fluorescence intensity value before administration F0)/fluorescence intensity value before administration F0×100%. The change of the intracellular calcium concentration indicated the function of TRP.

Inhibition on proliferation of human cancer cells: It is reported that the drugs that up-regulate TRPM8 or down-regulate TRPV1 can inhibit proliferation of breast cancer. To further investigate whether neferine follow the same way, human breast cancer cell line MDA-MB-231 and MDA-MB-453 as well as cancer gene-containing human breast gland epithelial cell line MCF10A-Myc and human pancreatic cancer cell line MIAPaCa-2 were employed and cultured in DMEM or DMEM/F12 medium with epithelial growth factor in vitro. After incubation with various concentrations of test agents for 72 h, the effective concentration of them for inhibiting the tumor cells was assayed according to the common method in the art.

Experiment data were represented with mean ±SD (X±S). Multi sample comparing uses one-way ANOVAs and double sample comparing uses t-test. Statistical difference was considered when P-value <0.05.

### EXAMPLE 1

### Effect of Neferine on TRPM8 mRNA Expression in DRG Cells under Normal Ambient Temperature (37° C.)

The purified culture medium of DRG cells was added with three different concentrations of test compounds and cultured at 37° C. in 5% CO₂ for 24 h. The total RNA was extracted immediately after culture. The TRPM8 agonist hexahydrothymol was positive control group and the normal culture medium was vehicle control group. Then the real-time PCR amplification was performed and TRPM8 mRNA was determined. As indicated in table 1, neferine could up-regulate the expression levels of TRPM8 mRNA dose-dependently (correlation coefficient 0.98); the effect was more significant when the dose was more than 5 μmol/L (i.e. 10 μmol/L).

TABLE 1
Effect of neferine on TRPM8 mRNA expression at 37° C. (x ± s)
(n = 8)

| | Concentrations | TRPM8 mRNA |
|---|---|---|
| Groups | (μmol/L) | expression (2^{-ΔΔCT}) |
| Vehicle control (37° C.) | — | 1.10 ± 0.21 |
| Neferine | 10 | 1.28 ± 0.27 |
| | 5 | 1.14 ± 0.31 |
| | 2.5 | 1.10 ± 0.27 |
| Positive control | 150 | 1.29 ± 0.31 |
| (hexahydrothymol) | 75 | 1.20 ± 0.28 |
| | 37.5 | 1.13 ± 0.22 |

### EXAMPLE 2

### Effect of Neferine on TRPM8 mRNA Expression in DRG Cells under High Temperature (39° C.)

The previous studies had shown that high temperature (e.g. 39° C.) could inhibit TRPM8 expression. The purified culture medium of DRG cells was added with three different concentrations of test compounds and cultured at 37° C. in 5% CO₂ for 23 h followed by 39° C. in 5% CO₂ for 1 h. The total RNA was extracted immediately after culture. The TRPM8 agonist hexahydrothymol was positive control group and the normal culture medium was vehicle control group. Then the real-time PCR amplification was performed and TRPM8 mRNA was determined. As indicated in table 2, neferine did not up-regulate TRPM8 mRNA expression.

TABLE 2
Effect of neferine on TRPM8 mRNA expression at 39° C. (x ± s)
(n = 8)

| | Concentrations | TRPM8 mRNA |
|---|---|---|
| Groups | (μmol/L) | expression (2^{-ΔΔCT}) |
| Vehicle control (37° C.) | — | 1.02 ± 0.25 |
| Vehicle control (39° C.) | — | 0.68 ± 0.20^{#} |
| Neferine | 10 | 0.79 ± 0.24 |
| | 5 | 0.78 ± 0.33 |
| | 2.5 | 0.74 ± 0.23 |
| Positive control | 150 | 0.96 ± 0.32 |
| (hexahydrothymol) | 75 | 0.91 ± 0.27 |
| | 37.5 | 0.89 ± 0.25 |

### EXAMPLE 3

### Effect of Neferine on TRPM8 mRNA Expression in DRG Cells under Low Temperature (19° C.)

The previous studies had shown that low temperature could improve TRPM8 expression. The purified culture medium of DRG cells was added with three different concentrations of test compounds and cultured at 37° C. in 5% CO₂ for 22 h followed by 19° C. in 5% CO₂ for 2 h. The total RNA was extracted immediately after culture. The TRPM8 agonist hexahydrothymol was positive control group and the normal culture medium was vehicle control group. Then the real-time PCR amplification was performed and TRPM8 mRNA was determined. As indicated in table 3, neferine could up-regulate the expression levels of TRPM8 mRNA dose-dependently (correlation coefficient 0.98). TRPM8 agonist hexahydrothymol also could up-regulate expression levels of TRPM8 mRNA dose-dependently.

TABLE 3
Effect of neferine on TRPM8 mRNA expression at 19° C. (x ± s)
(n = 8)

| | Concentrations | TRPM8 mRNA |
|---|---|---|
| Groups | (μmol/L) | expression (2^{-ΔΔCT}) |
| Vehicle control (37° C.) | — | 1.03 ± 0.23 |
| Vehicle control (19° C.) | — | 2.87 ± 0.90^{#} |
| Neferine | 10 | 3.49 ± 0.82 |
| | 5 | 3.37 ± 0.87 |
| | 2.5 | 3.25 ± 0.65 |
| Positive control | 150 | 4.07 ± 1.12* |
| (hexahydrothymol) | 75 | 3.72 ± 1.09 |
| | 37.5 | 3.67 ± 0.93 |

### EXAMPLE 4

### Effect of Neferine on Ca²⁺ in DRG Cells at Ambient Temperature (37° C.)

According to the protocol of example 1, the purified culture medium of DRG cells was added with three different concentrations of test compounds and cultured at 37° C. in 5% CO₂ for 24 h. The medium was discarded and the fluorescent dye was added and loaded for 30 min. The change of fluorescence intensity in cell was determined in a closed chamber on a microscope stage and the change of the intracellular calcium concentration was assayed. As indicated in table 4, neferine could increase the intracellular calcium concentration dose-dependently as same as TRPM8 agonist hexahydrothymol (correlation coefficient is 0.86 and 0.94). The effect was more significant when the dose is more than 5 μmol/L (i.e. 10 μmol/L).

TABLE 4
Effect of neferine on the function of TRPV1 (change of intracellular
calcium) at normal ambient temperature (37° C.)

| | Concentrations | Intracellular Calcium |
|---|---|---|
| Groups | (μmol/L) | changes [(F₁ − F₀)/F₀/%] |
| Vehicle control (37° C.) | — | 117.05 ± 17.28 |
| Neferine | 10 | 132.07 ± 19.30* |
| | 5 | 128.16 ± 11.48 |
| | 2.5 | 121.41 ± 10.05 |
| Positive control | 150 | 120.65 ± 12.19 |
| (hexahydrothymol) | 75 | 117.46 ± 13.58 |
| | 37.5 | 116.52 ± 10.22 |

### EXAMPLE 5

### Effect of Neferine on Ca²⁺ in DRG Cells at High Temperature (39° C.)

According to the protocol in example 2, the purified culture medium of DRG cells was added with three different concentrations of test compounds and cultured at 37° C. in 5% CO₂ for 22 h followed by 2 h at 39° C. (the experiment data of the peak value of calcium change after stimulated under high temperature was not shown here). The medium was discarded and the fluorescent dye was added and loaded for 30 min. The change of fluorescence intensity in cell was determined in a closed chamber on a microscope stage and the change of the intracellular calcium concentration was assayed. As indicated in table 5, the change of intracellular calcium concentration was inhibited after high temperature treatment. The test compound could increase the concentration of intracellular calcium dose-dependently (correlation coefficient >0.93) as same as TRPM8 agonist hexahydrothymol. The effect was more significant when the dose was more than 5 μmol/L (i.e. 10 μmol/L).

TABLE 5
Effect of neferine on the function of TRPV1 (change of intracellular
intracellular calcium) at high temperature (39° C.)

| | Concentration | Intracellular Calcium |
|---|---|---|
| Groups | (μmol/L) | changes [(F₁ − F₀)/F₀/%] |
| Vehicle control (37° C.) | — | 121.14 ± 12.09 |
| Vehicle control (39° C.) | — | 101.50 ± 9.82 |
| Neferine | 10 | 112.72 ± 11.74* |
| | 5 | 108.09 ± 12.85 |
| | 2.5 | 107.88 ± 13.07 |
| Positive control | 150 | 112.92 ± 13.19 * |
| (hexahydrothymol) | 75 | 108.63 ± 15.17 |
| | 37.5 | 107.54 ± 13.09 |

### EXAMPLE 6

### Effect of Neferine on TRPV1 mRNA Expression in DRG Cells at Normal Ambient Temperature (37° C.)

The purified culture medium of DRG cells was added with three different concentrations of test compounds and cultured at 37° C. in 5% CO₂ for 24 h. The total RNA was extracted immediately after culture. The TRPM8 agonist hexahydrothymol was positive control group and the normal culture medium was vehicle group. Then the real-time PCR amplification was performed and TRPV1 mRNA was determined. As indicated in table 6, neferine and hexahydrothymol could not change expression levels of TRPV1 mRNA under the test concentrations.

TABLE 6
Effect of neferine on TRPV1 mRNA expression in DRG cells
at 37° C. (x ± s) (n = 8)

| | Concentrations | TRPV1 mRNA |
|---|---|---|
| Groups | (μmol) | expression (2^{-ΔΔCT}) |
| Vehicle control (37° C.) | — | 1.02 ± 0.27 |
| Neferine | 10 | 1.04 ± 0.43 |
| | 5 | 1.09 ± 0.30 |
| | 2.5 | 0.96 ± 0.41 |
| Positive control | 150 | 1.08 ± 0.31 |
| (hexahydrothymol) | 75 | 1.10 ± 0.28 |
| | 37.5 | 0.96 ± 0.21 |

### EXAMPLE 7

### Effect of Neferine on TRPV1 mRNA Expression in DRG Cells at High Temperature (39° C.)

The previous studies had shown that high temperatures could improve TRPV1 expression. The purified culture medium of DRG cells was added with three different concentrations of test compounds and cultured at 37° C. in 5% CO₂ for 23 h followed by 39° C. in 5% CO₂ for 1 h (this is the appropriate stimulation time for 39° C., and the experiment data was not shown). The total RNA was extracted immediately after culture. The TRPM1 antagonist hexahydrothymol was positive control group and the normal culture medium was vehicle group. Then the real-time PCR amplification was performed and TRPV1 mRNA was determined. As shown in table 7, neferine could decrease the expression levels of TRPV1 mRNA dose-dependently (correlation coefficient −0.99). The effect was more significant when the dose was more than 2.5 μmol/L (i.e. 5 μmol/L).

TABLE 7
Effect of neferine on TRPV1 mRNA expression in
DRG cells at 39° C. (x ± s) (n = 8)

| | Concentrations | TRP V1 mRNA |
|---|---|---|
| Groups | (μmol/L) | expression (2^{-ΔΔCT}) |
| Vehicle control (37° C.) | — | 1.01 ± 0.24 |
| Vehicle control (39° C.) | — | 4.44 ± 0.75 |
| Neferine | 10 | 3.13 ± 0.51 |
| | 5 | 3.70 ± 0.69 |
| | 2.5 | 4.16 ± 0.71 |
| Positive control | 150 | 4.05 ± 0.81 |
| (hexahydrothymol) | 75 | 4.01 ± 0.73 |
| | 37.5 | 4.22 ± 0.49 |

### EXAMPLE 8

### Effect of Neferine on TRPV1 mRNA Expression in DRG Cells at Low Temperature (19° C.)

The previous studies had shown that low temperatures (e.g. 19° C.) could inhibit TRPV1 expression. The purified culture medium of DRG cells was added with three different concentrations of test compounds and cultured at 37° C. in 5% CO₂ for 22 h followed by at 19° C. in 5% CO₂ for 2 h (this is the appropriate stimulation time for 19° C., and the experiment data was not shown). The total RNA was extracted immediately after culture. The TRPM1 antagonist hexahydrothymol was positive control group and the normal culture medium was vehicle control group. Then the real-time PCR amplification was performed and TRPV1 mRNA was determined. As shown in table 8, neferine could further inhibit expression levels of TRPV1 mRNA dose-dependently (correlation coefficient −0.87) as same as hexahydrothymol.

TABLE 8
Effect of neferine on TRP V1 mRNA expression at 19° C. (x ± s)
(n = 8)

| | Concentrations | TRP V1 mRNA |
|---|---|---|
| Group | (μmol/L) | expression(2^{-ΔΔCT}) |
| Vehicle control (37° C.) | — | 1.01 ± 0.21 |
| Vehicle control (19° C.) | — | 0.61 ± 0.17^{#} |
| Neferine | 10 | 0.45 ± 0.23 |
| | 5 | 0.47 ± 0.20 |
| | 2.5 | 0.54 ± 0.17 |
| Positive control | 150 | 0.47 ± 0.15 |
| (hexahydrothymol) | 75 | 0.48 ± 0.19 |
| | 37.5 | 0.54 ± 0.17 |

### EXAMPLE 9

### Effect of Neferine on Ca²⁺ in DRG Cells at Normal Ambient Temperature (37° C.)

According to the protocol in example 1, the purified culture medium of DRG cells was added with three different concentrations of test compounds and cultured at 37° C. in 5% CO₂ for 24 h. The medium was discarded and the fluorescent dye was added and loaded for 30 min. The change of fluorescence intensity in cell was determined in a closed chamber on a microscope stage and the change of the intracellular calcium concentration was assayed. As shown in table 8, neferine could decrease the concentration of intracellular calcium dose-dependently (correlation coefficient is −0.85).

TABLE 9
Effect of neferine on the function of TRPV1 (change of intracellular
calcium) at normal ambient temperature (37° C.)

| | Concentrations | Intracellular Calcium |
|---|---|---|
| Groups | (μmol/L) | changes [(F₁ − F₀)/F₀/%] |
| Vehicle control (37° C.) | — | 104.38 ± 14.20 |
| Neferine | 10 | 96.33 ± 8.96 |
| | 5 | 98.64 ± 12.77 |
| | 2.5 | 98.15 ± 10.71 |
| Positive control | 150 | 98.31 ± 12.17 |
| (hexahydrothymol) | 75 | 101.82 ± 14.89 |
| | 37.5 | 100.14 ± 11.26 |

### EXAMPLE 10

### Effect of Neferine on Ca²⁺ in DRG Cells at High Temperature (39° C.)

According to the protocol in example 2, the purified culture medium of DRG cells was added with three different concentrations of test compounds and cultured at 37° C. in 5% CO2 for 22 h followed by 39° C. for 2 h (the experiment data of the peak value of calcium change after stimulated under high temperature was not shown here). The medium was discarded and the fluorescent dye was added and loaded for 30 min. The change of fluorescence intensity in cell was determined in a closed chamber on a microscope stage and the change of the intracellular calcium concentration was assayed. As shown in table 9, the change of intracellular calcium concentration could be activated after high temperature treatment. Neferine could inhibit the change of the intracellular calcium concentration dose-dependently (correlation coefficient −0.815). The effect was more significant when the dose was more than 2.5 μmol/L (i.e. 10 μmol/L).

TABLE 10
Effect of neferine on the function of TRPV1 (change of
intracellular calcium) at high temperature (39° C.)

| | Concentrations | Intracellular Calcium |
|---|---|---|
| Goups | (μmol/L) | changes [(F₁ − F₀)/F₀/%] |
| Vehicle control (37° C.) | — | 107.91 ± 14.05 |
| Vehicle control (39° C.) | — | 132.07 ± 16.83^{##} |
| Neferine | 10 | 119.49 ± 11.21 |
| | 5 | 120.51 ± 11.29 |
| | 2.5 | 128.63 ± 14.40 |
| Positive control | 150 | 126.08 ± 12.48 |
| (hexahydrothymol) | 75 | 132.01 ± 14.50 |
| | 37.5 | 133.49 ± 15.18 |

### EXAMPLE 11

### Effect of Neferine on Ca²⁺ in DRG Cells at Low Temperature (19° C.)

According to the protocol in example 1, the purified culture medium of DRG cells was added with three different concentrations of test compounds and cultured at 37° C. in 5% CO₂ for 21 h followed by 3 h at 19° C. (this is the appropriate stimulation time for the intracellular calcium change after stimulation under 19° C., and the experiment data was not shown). The medium was discarded and the fluorescent dye was added and loaded for 30 min. The change of fluorescence intensity in cell was determined in a closed chamber on a microscope stage and the change of the intracellular calcium concentration was assayed. As shown in table 11, the change of the intracellular calcium concentration could be inhibited after low temperature treatment. Neferine could further inhibit the change of the intracellular calcium concentration dose-dependently (correlation coefficient −0.99).

TABLE 11
Effect of neferine on the function of TRPV1 (change of
intracellular calcium) at low temperature (19° C.)

| | Concentrations | Intracellular Calcium |
|---|---|---|
| Groups | (μmol/L) | changes [(F₁ − F₀)/F₀/%] |
| Vehicle control (37° C.) | — | 108.43 ± 12.80 |
| Vehicle control (19° C.) | — | 99.84 ± 11.35^{#} |
| Neferine | 10 | 93.02 ± 10.54 |
| | 5 | 98.30 ± 11.89 |
| | 2.5 | 99.71 ± 10.96 |
| Positive control | 150 | 95.48 ± 10.23 |
| (hexahydrothymol) | 75 | 96.75 ± 11.08 |
| | 37.5 | 96.91 ± 11.34 |

### EXAMPLE 12

### Inhibition of Neferine on Human Cancer Cells

According to the “Inhibition on proliferation of human cancer cells” described above, the effective concentrations of neferine for the inhibition of tumors were obtained (table 11). A strong inhibition of neferine on breast cancer cells with a concentration below 2.1 μg/ml was shown and a inhibition concentration below 4 μg/ml was shown for pancreas.

TABLE 12
Inhibition of neferine on human cancer cells

| | Inhibition |
|---|---|
| | concentration |
| Human cancer cell lines | (μg/ml) |
| Human breast cancer cell line | 1.30 |
| MDA-MB-231 | |
| Human breast cancer cell line | 2.05 |
| MDA-MB-453 | |
| Cancer gene-containing human breast | 5.95 |
| epithelial cell line MCF10A-Myc | |
| Human pancreas cancer cell line | 3.64 |
| MIAPaCa-2 | |

### EXAMPLE 13

According to the method commonly known in the art, 35 g of commercial neferine was added into three times auxiliary material such as calcium carbonate and the mixture was granulated or directly pressed into tablets or coated to form 1000 tablets with each tablet containing 35 mg of neferine. Alternatively, the mixture can be put into 1000 hard capsules with each containing 35 mg of neferine. The above-mentioned preparations may be administrated orally as TRPM8 agonists for the treatment of chronic obstructive lung disease, Parkinson's disease, painful bladder syndrome, cold hypergesia, melanoma and prostate cancer and the like. Alternatively, they may also be used as TRPV1 antagonists for the treatment of pain, inflammation, schizophrenia, myasthenia syndrome, non-insulin-dependent diabetes, breast cancer and the like.

### EXAMPLE 14

According to the method commonly known in the art, 35 g of commercial neferine was added with polyoxyethylene hydrogenated castor oil and mixed to produce 1000 injections, or the mixture was further merged into injection saline to produce 1000 injections with each containing 35 mg of neferine. The above-mentioned preparations may be used as TRPM8 agonists for the treatment of chronic obstructive lung disease, Parkinson's disease, painful bladder syndrome, cold hyperalgesia, melanoma, prostate cancer. Alternatively, they may also be used as TRPV1 antagonists for the treatment of pain, inflammation, schizophrenia, myasthenia syndrome, non-insulin-dependent diabetes, breast cancer and the like.

### EXAMPLE 15

According to the method commonly known in the art, the neferine may be prepared into emplastrum with appropriate matrix and material for the skin application to create corresponding topical or general effective.

### REFERENCES

Kiselyov K., Soyombo A., Muallem S.: TRPpathies. J. Physiol. 2007; 578: 641-6530.

Nilius B., Owsianik G., Voets T., Peters J A.: Transient receptor potential cation channels in disease. Physiol. Rev. 2007; 87: 165-217.

Abramowitz J., Birnbaumer L.: Physiology and pathophysiology of canonical transient receptor potential channels. FASEB J, October 2008; 10.1096/fj.08-119495.

Sabnis A S., Shadid M., Yost G S., Reilly C A.: Human lung epithelial cells express a functional cold-sensing TRPM8 variant. Am. J. Respir. Cell Mol. Biol. 2008; 39: 466-474.

Yamamura H., Ugawa S., Ueda T., Morita A., Shimada S.: TRPM8 activation suppresses cellular viability in human melanoma. Am J Physiol Cell Physiol. 2008; 295: C296-C301.

Ashinger E S R., Steiginga M S., Hieble J P., Leon L A., Gardner S D., Nagilla R., Davenport E A., Hoffman B E., Laping N J., Su X.: AMTB, a TRPM8 channel blocker: evidence in rats for activity in overactive bladder and painful bladder syndrome. Am J Physiol Renal Physiol. 2008; 295: F803-F810.

Szallasi A., Cortright D N., Blum C A., Eid S R.: The vanilloid receptor TRPV1: 10 years from channel cloning to antagonist proof of concept. Nature Rev. Drug Discovery 2007, 6 (5) : 357-372.

Okuhara D Y., Hsia A Y., Xie M.: Transient receptor potential channels as drug targets. Expert Opinion on Therapeutic Targets 2007; 11 (3) :391-401.

1) Use of neferine in the preparation of medicaments of compounds for the up-regulation of TRPM8 in mammalians, wherein the medicaments do not comprise antitumor drugs.

2) The use claimed in claim 1 comprising the use in the preparation of medicaments for the prevention and/or treatment of chronic obstructive lung disease, Parkinson's disease, painful bladder syndrome, hypersensitivity to cold.

3) Use of neferine for the preparation of medicaments of compounds for the down-regulation of TRPV1 in mammalians, wherein the medicaments do not comprise antitumor drugs.

4) The use claimed in claim 3 comprising the use in the preparation of medicaments for the prevention and/or treatment of hyperalgesia to heat, allodynia, migraine, tooth pain, vulvodynia, painful gastrointestinal disease, esophagitis, gastro-esophageal reflux, functional bowel disorders, ulcerative colitis, pancreatitis, cystitis, hyper-reactivity of bladder, osteoarthritis, cough, chronic obstructive lung disease, asthma, allergic rhinitis, non-insulin-dependent diabetes, myasthenia syndrome, schizophrenia.

5) (canceled)

6) (canceled)

7) (canceled)

8) (canceled)

9) (canceled)

10) The use claimed in claim 1, wherein the compounds are further combined with pharmaceutically acceptable carriers.

11) The use claimed in claim 10, wherein the carriers include diluents, adhesives, absorbents, disintegrants, dispersants, wetting agents, auxiliary solvent, buffers and surfactants.

12) The use claimed in claim 2, wherein the compounds are further combined with pharmaceutically acceptable carriers.

13) The use claimed in claim 12, wherein the carriers include diluents, adhesives, absorbents, disintegrants, dispersants, wetting agents, auxiliary solvent, buffers and surfactants.

14) The use claimed in claim 3, wherein the compounds are further combined with pharmaceutically acceptable carriers.

15) The use claimed in claim 14, wherein the carriers include diluents, adhesives, absorbents, disintegrants, dispersants, wetting agents, auxiliary solvent, buffers and surfactants.

16) The use claimed in claim 4, wherein the compounds are further combined with pharmaceutically acceptable carriers.

17) The use claimed in claim 16, wherein the carriers include diluents, adhesives, absorbents, disintegrants, dispersants, wetting agents, auxiliary solvent, buffers and surfactants.

18) The use claimed in claim 1, wherein the mammalian is human.

19) The use claimed in claim 2, wherein the mammalian is human.

20) The use claimed in claim 3, wherein the mammalian is human.

21) The use claimed in claim 4, wherein the mammalian is human.

22) The use claimed in claim 1, wherein the neferine was prepared in powder, liquid and gas dosage form.

23) The use claimed in claim 22, wherein the dosage form include intestinal tract, oral, intramuscular, subcutaneous, nasal cavity, oral mucosa, skin, rectum and peritoneal administration dosage form.

24) The use claimed in claim 2, wherein the neferine was prepared in powder, liquid and gas dosage form.

25) The use claimed in claim 24, wherein the dosage form include intestinal tract, oral, intramuscular, subcutaneous, nasal cavity, oral mucosa, skin, rectum and peritoneal administration dosage form.

26) The use claimed in claim 3, wherein the neferine was prepared in powder, liquid and gas dosage form.

27) The use claimed in claim 26, wherein the dosage form include intestinal tract, oral, intramuscular, subcutaneous, nasal cavity, oral mucosa, skin, rectum and peritoneal administration dosage form.

28) The use claimed in claim 4, wherein the neferine was prepared in powder, liquid and gas dosage form.

29) The use claimed in claim 28, wherein the dosage form include intestinal tract, oral, intramuscular, subcutaneous, nasal cavity, oral mucosa, skin, rectum and peritoneal administration dosage form.
